(19) United States
(12) Patent Application Publication
(10) Pub. No.: US 2005/0090652 A1
(43) Pub. Date: Apr. 28, 2005

(54) Highly homogenous serum albumin as chiral selector

(76) Inventors: Carlo Bertucci, Pisa (IT); Guy Felix, La Seyne sur Mer (FR)

Correspondence Address: SYNNESTVEDT & LECHNER, LLP, 2600 ARAMARK TOWER, 1101 MARKET STREET, PHILADELPHIA, PA, 191072950, US

(21) Appl. No.: 10/501,067

(22) PCT Filed: Jan. 14, 2003
(86) PCT No.: PCT/GB03/00119

(30) Foreign Application Priority Data
Jan. 15, 2002 (GB) 0200816.7

(51) Int. Cl. G01N 33/53; C07D 311/02; C07D 239/02

(52) U.S. Cl. 530/417; 540/500; 562/401; 549/283; 544/309; 435/7.1

(57) ABSTRACT

The present invention provides the use of highly homogeneous serum albumin as a chiral selector, preferably immobilised, such as in enantioselective chromatography. The present invention also provides an enantioselective chromatography column comprising, as the immobilised phase, highly homogeneous serum albumin. The present invention also provides a process for selecting an enantiomer of a chiral compound, comprising the steps of exposing a mixture of enantiomers of the compound to highly homogeneous serum albumin for a period to allow selective binding of one enantiomer to the albumin; and separating the relatively unbound enantiomer from the albumin.

The present invention relates to separation techniques.

Serum albumin has been successfully used as a chiral selector in enantioselective chromatography, such as high-performance liquid chromatography (HPLC). Albumin can act as a chiral stationary phase (CSP) when immobilised (Domenici et al, 1990, Chromatographia, 29, 170-176; Bertucci & Domenici, 2002, Current Medicinal Chemistry, 9, 1463-1481; Wang Pen-Cheng et al, 2002, Journal of Chromatography, 942(1-2), 115-22; Kato Masaru et al, 2002, Analytical Chemistry, 74 (8), 1915-21; Tao & Gilpin, 2001, Journal of Chromatographic Science, 39(5), 205-12;), for example, on agarose or silica with, for example, glutaraldehyde (Andersson et al, 1992, J. Chromalogr., 591, 65-73). Using such a system, Yang and Hage (1993, J. Chromatogr., 645, 241-250) demonstrated that the L-enantiomer of tryptophan has higher affinity, whereas Lagercrantz et al (1981, Comp. Biochem. Physiol., 69C, 375-378) have used this system to separate chiral forms of warfarin. Racemic mixtures of aryl propionate anti-inflammatory drugs (Noctor et al, 1991, Chromatographia, 31, 55-69) and N-methylated barbiturates (Krug et al, 1994, Arzneim.-Forsch., 44, 109-113) have also been resolved.

The application of serum albumin as a chiral selector has been extended to use in either the liquid or solid phase in applications such as capillary electrophoresis and affinity capillary electrophoresis (Gotti et al, 2001, Journal of Pharmaceutical and Biomedical Analysis, 24(5-6), 863-70; Haginaka, 2001, Journal of Chromatography, 906(1-2), 253-73; Chiari et al, 2000, Electrophoresis, 21(12), 2343-51; Haginaka, 2000, Journal of Chromatography, 875(1-2), 235-54; Ding et al, 1999, Electrophoresis, 20(9), 1890-4; Birnbaum and Nilsson, 1992, Anal. Chem., 64, 2872-2874; Arai et al, 1994, Anal. Biochem., 217, 7-11). For a review of the use of capillary electrophoresis to separate enantiomers of chiral compounds, see Lloyd et al (1997, J. Chromatogr. A., 792(1-2), 349-69).

The serum albumin-based columns used in these methods have had the problem of significant variation of chromatographic performance, including variation in the retention of enantiomers and in enantioselectivity. The variable efficacy of previously available methods has made the development and validation of HPLC methods difficult.

Modification of methods utilising immobilised albumin as a CSP have involved the introduction of additives into the mobile phase. For example, Hayball et al (1994, J. Chromatogr. B, 662, 128-133) describes how the separation (R)- and (S)-enantiomers of ketorolac using human serum albumin (HSA)-CSP can be modified by the addition of octanoic acid into the mobile phase to increase the ratio of percentage unbound (S)- to unbound (R)-enantiomer. Domenici et al, 1991, J. Phar. Sci., 80, 164-166, showed that enantioselectivity in the resolution of rac-lorazepam can be significantly increased by adding (S)-warfarin in the mobile phase. These documents suggest that changes in the performance of CSP albumin as a chiral separator can be optimised by modification of the mobile phase.

The present invention addresses the problem of variable chromatographic performance. Additionally, the present invention provides a method that can yield higher levels of enantioselectivity.

According to a first aspect of the invention, there is provided the use of highly homogeneous serum albumin as a chiral selector. Preferably the highly homogeneous serum albumin is immobilised.

The term “serum albumin” refers generically to serum-derived albumin and/or recombinantly produced albumin. Preferably, the term “serum albumin” includes within its meaning variant albumin. A “variant albumin” refers to an albumin protein wherein at one or more positions there have been amino acid insertions, deletions, or substitutions, either conservative or non-conservative, provided that such changes result in an albumin protein capable of acting as a chiral selector, as defined below. Typically a variant albumin also substantially retains other basic properties of albumin. For example, a variant albumin may have a binding activity (type of and specific activity), thermostability and/or activity in a certain pH-range (pH-stability) which has not significantly been changed from an albumin protein with the original sequence. “Significantly” in this context means that one skilled in the art would say that the properties of the variant may still be different but would not be unobvious over the ones of the original protein.

By “conservative substitutions” is intended combinations such as Gly, Ala; Val, Ile, Leu; Asp, Glu; Asn, Gln; Ser, Thr; Lys, Arg; and Phe, Tyr. Such variants may be made using the methods of protein engineering and site-directed mutagenesis known in the art, such as those disclosed in U.S. Pat. No. 4,302,386 issued 24 Nov. 1981 to Stevens, incorporated herein by reference.

Typically an albumin variant will have more than 40%, usually at least 50%, more typically at least 60%, preferably at least 70%, more preferably at least 80%, yet more preferably at least 90%, even more preferably at least 95%, most preferably at least 98% or more sequence identity with naturally occurring albumin. The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally. The alignment may alternatively be carried out using the Clustal W program (Thompson et al., 1994). The parameters used may be as follows:

Fast pairwise alignment parameters: K-tuple(word) size; 1, window size; 5, gap penalty; 3, number of top diagonals; 5. Scoring method: x percent.

Multiple alignment parameters: gap open penalty; 10, gap extension penalty; 0.05.

Scoring matrix: BLOSUM.

Albumin may be obtained from any source. Typically the source is mammalian. In one preferred embodiment the serum albumin is human serum albumin. The term “human serum albumin” includes the meaning of a serum albumin having an amino acid sequence naturally occurring in humans, and variants thereof as defined above. Preferably the albumin has the amino acid sequence disclosed in WO 90/13653 or a variant thereof. The term “human serum albumin” also includes the meaning of fragments of full-length human serum albumin or variants thereof. Any fragment may be used, so long as it retains the ability to act as a chiral selector. A fragment will typically be at least 50 amino acids long. A fragment may comprise at least one whole sub-domain of albumin. Domains of human albumin have been expressed as recombinant proteins (Dockal, M. et al., 1999, J. Biol. Chem., 274, 29303-29310), where domain I was defined as consisting of amino acids 1-197, domain II was defined as consisting of amino acids 189-385 and domain III was defined as consisting of amino acids 381-585. Partial overlap of the domains occurs because of the extended α-helix structure (h10-h1) which exists between domains I and II, and between domains II and III (Peters, 1996, op. cit., Table 2-4). Human albumin also comprises six sub-domains (sub-domains IA, IB, IIA, IIB, IIIA and IIIB). Sub-domain IA comprises amino acids 6-105, sub-domain IB comprises amino acids 120-177, sub-domain IIA comprises amino acids 200-291, sub-domain IIB comprises amino acids 316-369, sub-domain IIIA comprises amino acids 392-491 and sub-domain IIIB comprises amino acids 512-583. A fragment may comprise a whole or part of one or more domains or sub-domains as defined above, or any combination of those domains and/or sub-domains.

In another preferred embodiment the serum albumin is bovine serum albumin. The term “bovine serum albumin” includes the meaning of a serum albumin having an amino acid sequence naturally occurring in cows, and variants thereof as defined above. The term “bovine serum albumin” also includes the meaning of fragments of full-length bovine serum albumin or variants thereof. Any fragment may be used, so long as it retains the ability to act as a chiral selector. BSA comprises domains and sub-domains equivalent to those defined above for HSA. A fragment of BSA may comprise a whole or part of one or more domains and/or sub-domains, or any combination of those domains or sub-domains.

In another preferred embodiment the serum albumin is serum albumin derived from (i.e. has the sequence of) one of serum albumin from dog, pig, goat, turkey, baboon, cat, chicken, ovalbumin (e.g. chicken ovalbumin), donkey, guinea pig, hamster, horse, rhesus monkey, mouse, pigeon, rabbit, rat and sheep and includes variants and fragments thereof as defined above that retain the ability to act as a chiral selector.

Therefore, the serum albumin may have the sequence of cow albumin, for example as taken from Swissprot accession number P02769.

The serum albumin may have the sequence of dog albumin, for example as taken from Swissprot accession number P49822.

The serum albumin may have the sequence of pig albumin, for example as taken from Swissprot accession number P08835.

The serum albumin may have the sequence of goat albumin, for example as available from Sigma as product no. A2514 or A4164.

The serum albumin may have the sequence of turkey albumin, for example as taken from Swissprot accession number O73860.

The serum albumin may have the sequence of baboon albumin, for example available from Sigma as product no. A1516.

The serum albumin may have the sequence of cat albumin, for example as taken from Swissprot accession number P49064.

The serum albumin may have the sequence of chicken albumin, for example as taken from Swissprot accession number P19121.

The serum albumin may have the sequence of chicken ovalbumin, for example as taken from Swissprot accession number P01012.

The serum albumin may have the sequence of donkey albumin, for example as taken from Swissprot accession number P39090.

The serum albumin may have the sequence of guinea pig albumin, for example as available from Sigma as product no. A3060, A2639, O5483 or A6539.

The serum albumin may have the sequence of hamster albumin, for example available from Sigma as product no. A5409.

The serum albumin may have the sequence of horse albumin, for example as taken from Swissprot accession number P35747.

The serum albumin may have the sequence of rhesus monkey albumin, for example as taken from Swissprot accession number Q28522.

The serum albumin may have the sequence of mouse albumin, for example as taken from Swissprot accession number O89020.

The serum albumin may have the sequence of pigeon albumin, for example as defined by Khan et al, 2002, Int. J. Biol. Macromol., 30(3-4),171-8.

The serum albumin may have the sequence of rabbit albumin, for example as taken from Swissprot accession number P49065.

The serum albumin may have the sequence of rat albumin, for example as taken from Swissprot accession number P36953.

The serum albumin may have the sequence of sheep albumin, for example as taken from Swissprot accession number P14639.

Many naturally occurring mutant forms of albumin are known. Many are described in Peters, (1996, op. cit., p.170-181). A variant as defined above may be one of these naturally occurring mutants.

Serum albumin, such as human, bovine, or any of the other serum albumin types defined above, for use in the present invention may be serum-derived and is therefore typically obtained by extraction from blood. Examples of extraction and separation techniques include those disclosed in: JP 03/258 728 on the use of a cation exchanger; EP 428 758 on the use of anion exchange; EP 452 753 on the use of heating, adding salt and diafiltering; and WO 96/37515 and WO 00/44772 on complex multi-stage purification processes.

Alternatively the serum albumin used in the present invention is recombinantly produced. Thus a polynucleotide encoding a serum albumin, such as a human or bovine serum albumin, may be transformed into a cell and expressed.

Many expression systems are known, including bacteria (for example E. coli and Bacillus subtilis), yeasts (for example Saccharomyces cerevisiae, Pichia pastoris and Kluyveromyces lactis), filamentous fungi (for example Aspergillus), plant cells, whole plants, animal cells and insect cells.

In one embodiment the preferred host cells are the yeasts Saccharomyces cerevisiae, Kluyveromyces lactis and Pichia pastoris. It is particularly advantageous to use a yeast deficient in one or more protein mannosyl transferases involved in O-glycosylation of proteins, for instance by disruption of the gene coding sequence.

The albumin protein sequence does not contain any sites for N-linked glycosylation and has not been reported to be modified, in nature, by O-linked glycosylation. However, it has been found that recombinant human serum albumin (rHA) produced in a number of yeast species can be modified by O-linked glycosylation, generally involving mannose. The mannosylated albumin is able to bind to the lectin Concanavalin A. The amount of mannosylated albumin produced by the yeast can be reduced by using a yeast strain deficient in one or more of the PMT genes (WO 94/04687). The most convenient way of achieving this is to create a yeast which has a defect in its genome such that a reduced level of one of the Pmt proteins is produced. For example, there may be a deletion, insertion or transposition in the coding sequence or the regulatory regions (or in another gene regulating the expression of one of the PMT genes) such that little or no Pmt protein is produced. Alternatively, the yeast could be transformed to produce an anti-Pmt agent, such as an anti-Pmt antibody.

If a yeast other than S. cerevisiae is used, disruption of one or more of the genes equivalent to the PMT genes of S. cerevisiae is also beneficial, eg in Pichia pastoris or Kluyveromyces lactis. The sequence of PMT1 (or any other PMT gene) isolated from S. cerevisiae may be used for the identification or disruption of genes encoding similar enzymatic activities in other fungal species. The cloning of the PMT1 homologue of Kluyveromyces lactis is described in WO 94/04687.

The yeast will advantageously have a deletion of the HSP150 and/or YAP3 genes as taught respectively in WO 95/33833 and WO 95/23857.

In a preferred embodiment the yeast is transformed with an expression plasmid based on the Saccharomyces cerevisiae 2 μm plasmid. At the time of transforming the yeast, the plasmid contains bacterial replication and selection sequences, which are excised, following transformation, by an internal recombination event in accordance with the teaching of EP 286 424. The plasmid also contains an expression cassette comprising: a yeast promoter (eg the Saccharomyces cerevisiae PRB1 promoter), as taught in EP 431 880; a sequence encoding a secretion leader which, for example, comprises most of the natural HSA secretion leader, plus a small portion of the S. cerevisiae α-mating factor secretion leader as taught in WO 90/01063; the HSA coding sequence, obtainable by known methods for isolating cDNA corresponding to human genes, and also disclosed in, for example, EP 73 646 and EP 286 424; and a transcription terminator, preferably the terminator from Saccharomyces ADH1, as taught in EP 60 057. Preferably, the vector incorporates at least two translation stop codons.

The choice of various elements of the plasmid described above is not thought to be directly relevant to the purity of the albumin product obtained, although the elements may contribute to an improved yield of product.

Techniques for the purification of serum and recombinantly expressed albumin are well known in the art.

An impure albumin solution may, for example, be obtained from serum by any of the plethora of extraction and purification techniques developed over the last 50 years, for example those disclosed in Stoltz et al (1991) Pharmaceut. Tech. Int. June 1991, 60-65 and More & Harvey (1991) in “Blood Separation and Plasma Fractionation” Ed. Harris, Wiley-Liss, 261-306. Alternatively an impure albumin solution may be obtained from the fermentation medium for a micro-organism transformed with a nucleotide sequence encoding the amino acid sequence of albumin; preferably separating the micro-organism from the fermentation medium; and conditioning the medium, if necessary, for further purification. In a further alternative, an impure albumin solution may be obtained, for example, from a transgenic animal, such as goat, sheep or cattle, from, for instance, the milk or the blood of the animal or, in the case of transgenic chicken, from the egg white. In a still further alternative, an impure albumin solution may be obtained from a transgenic plant, such as tobacco, potato or corn (maize).

Typically the process of obtaining highly pure albumin from an impure albumin solution comprises one or more of the following steps: exposing the impure albumin solution to three successive chromatography steps; ultrafiltering/diafiltering the product; passing the ultrafiltered product through a further chromatography step; ultrafiltering/diafiltering again before purification through two further chromatographic steps; and final ultrafiltration/diafiltration.

Preceding or following any of the above mentioned procedures the albumin solution may undergo buffer exchange, concentration, dilution, heating (including sterilisation), cooling or may have salts etc. added to the albumin solution which may, for example, condition or adjust the pH of the solution. Optionally, the albumin may be treated with a reducing agent or may undergo a decolouration step.

For an example of the purification of recombinantly expressed albumin, see EP 658 569, or preferably WO 96/37515 and WO 00/44772.

The final product may be formulated to give it added stability. Any stabilising agent known in the art may be used. Typically, octanoate, polysorbate 80 or N-acetyl tryptophan may be used as a stabilising agent.

Where a serum albumin is formulated with a stabilising agent, it is preferably subjected to defatting prior to its use according to the first aspect of the present invention. Any suitable process known in the art, such as Chen's charcoal process (Chen R F, 1967, J. Biol. Chem., 242, 173-181), may be used to defat a serum albumin formulated with a stabilising agent. For example, octanoate may be present at, at most, 40, 35, 30, 25, 20, 15, 10, 5, 3, 2, 1 or substantially 0 mM after defatting. Polysorbate 80 may be present at, at most, 20, 15, 10, 5, 3, 2, 1 or substantially 0 mg.mL⁻¹ after defatting. N-acetyltryptophan may be present, at most, at 80, 70, 670, 50, 40, 30, 10, 5, 2, 1 or substantially 0 nmole.g⁻¹ after defatting.

Using such production methods as described above it is possible to obtain highly homogenous serum albumin. Highly homogeneous albumin can be characterised by a number parameters. Highly homogeneous albumin will have at least one property selected from the group consisting of:
(i) extremely low levels of colorants. The term “colorant” as used herein means any compound which colours albumin. For example, a pigment is a colorant which arises from the organism, such as yeast, which is used to prepare recombinant albumin, whereas a dye is a colorant which arises from chromatographic steps to purify the albumin.
(ii) extremely low levels of, or be essentially free of, aluminium, lactate, citrate, metals, non-albumin human proteins, such as immunoglobulins, pre-kallikrein activator, transferrin, α₁-acid glycoprotein, haemoglobin and blood clotting factors, prokaryotic proteins, fragments of albumin, albumin aggregates or polymers, or endotoxin, bilirubin, haem, yeast proteins, animal proteins and viruses. By essentially free is meant below detectable levels.
(iii) at least 99.5% monomeric and dimeric, preferably essentially 100% monomeric and dimeric. Up to 0.5%, preferably 0.2% trimer is tolerable but larger forms of albumin are generally absent.
(iv) a nickel ion level of less than 100 ng, based on one gram of albumin, as measured by the method defined in WO 00/44772.
(v) a glycation level of less than 0.6, preferably less than 0.10, 0.075 or 0.05 moles hexose/mole protein as measured in the Amadori product assay, a microassay for glycated protein. The microassay measures the stable Amadori product (AP) form of glycated protein, by oxidation of the C-1 hydroxyl groups of AP with periodate. The formaldehyde released by periodate oxidation is quantitated by conversion to a chromophore, diacetyldihydrolutidine (DDL), by reaction with acetylacetone in ammonia. DDL is then detected colorimetrically. Samples are assayed after desalting using a Pharmacia PD-10 (G25 Sephadex) column and then the albumin in the samples is re-quantitated by the Bradford method and 10 mg albumin assayed. A fructose positive control is included, and the absorbances are read on a Shimadzu UV 2101 spectrophotometer at 412 nm. For every mole of hexose one mole of Amadori product is formed.
(vi) at least 90% or 95%, preferably at least 96%, more preferably at least 97%, even more preferably at least 98%, even more preferably at least 99%, most preferably substantially 100% of the albumin molecules have an intact C-terminus.
(vii) a content of Concanavalin A-binding albumin of less than 0.5% (w/w), preferably less than 0.3%, 0.2% or 0.15%. Concanavalin A (Con A) binds molecules which contain α-D-mannopyranosyl, α-D-glucopyranosyl and sterically related residues. Con A-binding can be assayed using Con A Sepharose (Pharmacia, Cat. No. 17-0440-01) affinity chromatography of serum albumin in order to determine the content of mannosylated albumin. Serum albumin is diluted to 5% (w/v) with 145 mM sodium chloride then 1:1 with Con A dilution buffer (200 mM sodium acetate, 85 mM sodium chloride, 2 mM magnesium chloride, 2 mM manganese chloride, 2 mM calcium chloride pH5.5). 100 mg serum albumin is then loaded onto an equilibrated 2 mL Con A Sepharose column which is then washed (5×4 mL) with Con A equilibration buffer (100 mM sodium acetate, 100 mM sodium chloride, 1 mM magnesium chloride, 1 mM manganese chloride, 1 mM calcium chloride pH5.5). The column is eluted with 6 mL Con A elution buffer (100 mM sodium acetate, 100 mM sodium chloride, 0.5M methyl-α-D-mannopyranoside pH5.5). Monomeric albumin in eluate (diluted as appropriate to make sure the sample falls in the middle of the standard is curve) are quantified by the Bradford method (Bradford, 1976, Anal. Biochem., 72, 248-254) using a 0-0.12 mg.mL⁻¹ albumin standard curve, and the Con A binding albumin monomer recovered in the eluate is expressed as a percentage of the load.
(viii) a free thiol content of at least 0.85, 0.8, 0.75, 0.7 or 0.65 mole SH/mole protein when measured by using Ellman's Reagent, 5,5′-dithiobis-(2-nitrobenzoate) (DTNB), which is a specific means of detecting free sulfhydryl groups such as cys-SH (Cys-residue 34 in the case of rHA). The reaction releases the 5-thio-2-nitrobenzoate ion TNB²⁻ which has an absorption maximum at 412 nm. By measuring the increase in absorbance at 412 nm and dividing by the molar extinction coefficient of the TNB²⁻ ion at 412 nm, the free sulfhydryl content of rHA can be calculated.
(ix) substantially no C18 or C20 fatty acids, when analysed by acidic solvent extraction and gas chromatography of free fatty acids using a C17:0 internal standard.
(x) a molecular weight distribution of at least 50, 60, 70, 80, 90, 95, 98, 99, 99.9 or substantially 100% of albumin molecules with a molecular weight spread no greater than 2000, 1500, 1000, 900, 800, 700, 600, 500, 400, 300, 200 or fewer daltons when determined by mass analysis using electrospray mass spectrometry. Protein samples are desalted employing standard methods such as dialysis or chromatography and exchanged or diluted into typically 50% (v/v) organic solvent, such as acetonitrile or methanol supplemented with acid, such as 0.1-10% (v/v) formic acid for positive ion electrospray or with base such as 0.1-10% (v/v) ammonium hydroxide for negative ion electrospray. Protein solutions at concentrations optimal for the employed ion source, typically 1-50 μM, are introduced into the electrospray ion source at appropriate flow rates of typically 0.01-100 μL.min⁻¹ using standard methods such as continuous flow, loop injection, or off-line, using a syringe pump, a HPLC pump or nanoelectrospray vial respectively. The instrument analyser/s are tuned for optimal transmission and resolution (the latter should exceed 500, as defined by baseline separation of 500-501 m/z) and calibrated using a local protocol and a suitable calibrant typically a protein (e.g. horse myoglobin) or a surfactant (e.g. PEG). Spectra are acquired, averaged and processed to subtract baseline noise, smooth signal, centroid peaks, measure mass and deconvolute data to a true mass scale using appropriate software known in the art and commercially available. One example protocol (Bertucci et al, 2001, Biochimica et Biophysica Acta, 1544, 386-392) utilises dilution in H₂O/CH₃CN 50:50 with 5% HCOOH, to a final albumin concentration of 40-50 μM and analysis by Ionspray Mass Spectrometry, carried out on a Perkin-Elmer SCIEX API III triple quadrupole mass spectrometer (Sciex, Thomill, Canada) equipped with an articulated Ionspray interface, operated using the following parameters: ionspray voltage 5.5 kV; orifice voltage 90 V; scan range 1400-2200 u; scan time 8.42 s; resolution>1 u. The spectra are acquired in Multichannel acquisition (MCA) mode summing 20 scans. Analysis is performed by continuous infusion into the source by a Harvard model 22 syringe pump (Harvard Apparatus, South Natick, Mass.) at 5 μl.min⁻¹ flow rate. All measurements are carried out at room temperature. For example, highly homogeneous HSA may have one of the above mentioned percentages of protein molecules within the range of 66400 to 67000 daltons. Typically, when analysed by one of the above method, the main peak obtained for a highly homogeneous serum albumin is usually within 0.05%, more usually within 0.01% of the theoretical mass (in the case of HSA, the theoretical mass is 66,438 daltons). In one embodiment, the mass profile is determined by electrospray mass spectrometry (ESI-MS) with a span of 1000 daltons either side of the main peak. The profile is scrutinised for the presence of micro-heterogeneity observed as discretely resolved components or alternatively as a broadening in the mass peak widths at half height. The relative abundance of resolved components may be measured as relative ion count and expressed as percentage (%) composition. The profile of highly homogeneous serum albumin as used in the present invention is typically at least 50%, 60%, 70%, 80%, 90% or more similar to native (unmodified) primary structure composition. Conversely, commercially available serum albumins as used in prior art columns are typically less than 50% unmodified native primary structure, the remainder accounted for by the presence of post-translationally processed albumin molecules including albumin with cysteinylated thiol, glycation and/or N-terminal and/or C-terminal degradation. The relative abundance of components in highly homogeneous serum albumin may be additionally measured using other quantitative techniques, including neutral coated capillary zone electrophoresis with detection by absorbance in the UV region (Denton & Harris, 1995, J. Chromatog. A., 705, 335-341).

The homogeneity of a population of serum albumin molecules, may be determined by electrophoretic and chromatographic techniques. For example, SDS PAGE may be performed using standard methods. Local protocols should employ PAGE gels capable of separating proteins within the 20-200 kDa molecular size range. Native PAGE is optimised to yield focusing and separation of proteins with differing mass and/or charge. Electrophoretically separated protein components are visualised by chemical staining methods (e.g. Coomassie Blue dyes), which should have detection limits of typically greater than 0.1 μg. Quantitation is achieved by subsequent densitometric absorbance scanning with calibration against protein standards.

Gel permeation chromatography is performed using a column, typically with a separation range of 10-500 kDa molecular size, and with analytical dimensions. An optimally buffered aqueous mobile phase is pumped using an HPLC system and eluting components are detected by absorbance in the UV region. Peak quantitation is facilitated by chromatogram integration and calibration against standards of the test serum albumin.

Usually, when analysed by SDS PAGE, native PAGE and gel permeation chromatography, a highly homogeneous serum albumin preparation will display one or two of the following features
(a) at least 99%, preferably 99.9%, of the protein molecules in the population will be serum albumin.
(b) no more than 10, 9, 8, 7, 6, 5, 4, 3, 2%, preferably no more than 1%, of albumin protein molecules in the population will be dimeric.

The homogeneity of population of serum albumin molecules, may also be analysed by electrospray mass spectrometry (ESMS) and by peptide mapping.

In a preferred embodiment, when analysed by ESMS and peptide mapping, a highly homogeneous serum albumin preparation will have the correct native primary sequence of full length HSA or a fragment thereof as defined above and will not have post-translational modifications.

Commercially available albumins, with the exception of Recombumin albumin (Delta Biotechnology, UK), are heterogeneous, showing a quite wide distribution of molecular weight. (“Recombumin” is a registered trademark.) This can result in differences in the binding properties of protein molecules within a population. In addition, heterogeneity of commercial albumin is also observed in the free thiol content (viii) and degree of glycation (v). All these aspects can change the binding properties of the protein. This can result in a low reproducibility of chromatographic performances, such as when used as a chiral selector.

In one embodiment, highly homogeneous serum albumin has at least two, three or four, of the features (v), (viii), (x) and (a) as defined above. One of the features may be feature (x) in combination with one, two or three of features (v), (viii) and (a).

Albumin can act as a chiral selector in the liquid or solid phase. In one embodiment, highly homogeneous serum albumin as used above can be used as a chiral selector in the liquid phase. For example, albumin can be used in the eluent of a thin layer chromatographic enantioseparation of compounds, using the method of Lepri et al (Journal of Planar Chromatography—Modern TLC, 6(2), 100-104, 1993).

Preferably the albumin is used as a chiral selector in the solid phase. Albumin may be immobilised on any suitable support. For example, a silica matrix may be used to immobilise the serum albumin (for example, see Tao & Gilpin, 2001, Journal of Chromatographic Science, 39(5), 205-12), such as an epoxy silica matrix like Kromasil 200 Å (5 μm) (i.e. 200 Å mean pore size between beads and 5 μm silica bead size) or Polygosil 300 Å (7 μm). Alternatively, serum albumin can be immobilised on agarose or a polymer matrix, such as polystyrene divinyl benzene (PSDB) or poly(vinylidene fluoride) (Wang Pen-Cheng et al, op. cit.). Albumin can be immobilised in a hydrogel, such as a tetramethoxysilane-based hydrogel (Kato Masaru et al, 2002, op. cit.).

Albumin can be immobilised by methods well known in the art. For example, methods for protein immobilisation are reviewed in Haginaka (2001, op. cit.), the teachings of which are incorporated herein by reference.

Serum albumin can be immobilised on silica gel, for example, by following the protocol provided in the examples. Other methods are known in the art. For example, see Bertucci et al (1999, Chirality, 11(9): 675-679).

Serum albumin can be immobilised on agarose activated with cyanogen bromide. 200 mg of CNBr/ml agarose will couple 5-10 mg/ml of albumin.

Whichever immobilisation technique is used, it is preferred that the thus immobilised albumin retains the binding properties of unbound (i.e. prior to immobilisation) albumin. An immobilised albumin can be said to retain the binding properties of unbound albumin if it can bind at least one, two, or all three of phenylbutazone, diazepam and bilirubin with the same capacity as unbound albumin. This is particularly important where the immobilised albumin is to be used for studying the binding of a drug to albumin, or interaction between a drug and albumin.

Albumin is “enantioselective” because it is able to bind with greater affinity to one enantiomer of at least some chiral compounds than to the other enantiomer. Typically, albumin, when exposed to a mixture of enantiomers of a chiral compound for a period to allow selective binding of one enantiomer to the albumin, will bind to one of the enantiomers with an affinity that is up to 10¹-fold, 10²-fold, 10³-fold, or more, greater than the affinity with which the other enantiomer is bound. For example, the affinity of L-tryptophan is 100-fold higher when compared to its antipode, and the affinity of (S)-oxazepam-hemisuccinate is 35-fold higher with respect to the (R)-enantiomer (Peters et al., op. cit.; Kragh-Hansen, U., 1990, Dan. Med. Bull., 37, 57-84).

The term “mixture of enantiomers” typically refers to a racemic mixture, although other proportions, such as about 99:1, 95:5, 90:10, 80:20, 70:30, 60:40, 55:45 and 51:49, as well as all proportions in between these values, are included in the meaning.

Albumin, when exposed to a mixture of enantiomers of a chiral compound, will selectively bind to one enantiomer under suitable conditions. The phrase “suitable conditions” includes in its meaning all conditions at which one enantiomer of the chiral compound has a higher binding affinity for the highly homogenous serum albumin than the other enantiomer. Optimal conditions can be established for each different chiral compound by routine testing. Typically, suitable conditions can be achieved by using a buffer, like phosphate buffer at pH 6-8 (preferably at about pH 7.4), with addition of an organic modifier to reduce the time of analysis. An example of a suitable organic solvent is 1-propanol because it does not denature the protein when used at concentrations up to 20%. Similarly, acetonitrile can be used in concentrations up 15%. As a non-limiting example, enantiomer separation can be performed by HPLC using highly homogeneous serum albumin immobilised on silica gel and a 1-propanol/phosphate buffer (pH 7.5) 15/85 (v/v) with a 0.6 ml/min flow rate.

The highly homogeneous serum albumin according to the first aspect of the invention can be used to separate enantiomers of any chiral compound that interact differentially with albumin. This can be readily tested, for example, by using circular dichroism to determine stereospecific binding of a chiral compound to albumin as described in the examples. However, highly homogeneous serum albumin will typically be useful to separate enantiomers of chiral compounds such as amino acids, amino acid derivatives (such as N-derivatised amino acids), sulfoxides and sulfoximine derivatives, racemic amines (e.g. prilocaine) non-steroidal anti-inflammatory drugs, aryl propionate anti-inflammatory drugs, N-methylated barbiturates, benzodiazepines, 2-arylpropionic acid derivatives (e.g. naproxen, flurbiprofen and other such profens) and coumarins. In particular, highly homogeneous serum albumin will typically be useful to separate enantiomers of chiral compounds such as warfarin (and its metabolites, such as 6- and 7-hydroxywarfarin and warfarin alcohols); benzodiazepines such as lorazepam (e.g. lorazepam hemisuccinate), oxazepam or temazepam; N-benzoyl-DL-leucine, amino acids such as tryptophan; benzoin, eprisone, chlorpheniramine, kynurenine, prilocaine, promethazine, donepezil and its salts (e.g. donepezil hydrochloride, sold under the trade name Asicept), thiopenfal, and non-steroidal anti-inflammatories such as profens (e.g. ibuprofen, naproxen, ketoprofen, suprofen or fenoprofen). For the avoidance of doubt, a chiral compound, the enantiomers of which are suitable for separation according to the present invention, may comprise more than one chiral centre and may, for example, be a diastereomer.

Thus highly homogeneous serum albumin as described above can be used as a chiral selector. Typically this involves the use of the serum albumin, immobilised to a solid support, in enantioselective chromatography, such as high performance liquid chromatography (HPLC) (Haginaka, 2001, op. cit.; Haginaka, 2000, op. cit.). However, other suitable techniques include capillary electrophoresis (CE) (Kato Masaru et al, 2002, op. cit.; Gotti et al, 2001, op. cit.; Chiari et al, 2000, op. cit.; Haginaka, 2000, op. cit.; Ding et al, 1999, op. cit.) . The use of highly homogeneous serum albumin as defined above in the mobile phase can improve the reproducibility of the selector performances, in the same way as discussed for HPLC.

In a preferred embodiment of the first aspect of the invention, the type of chromatography for which immobilised highly homogeneous serum albumin is used is high performance liquid-phase (HPLC) chromatography, although the immobilised highly homogeneous serum albumin can also be used in other forms of chromatography, such a ‘normal pressure’ liquid chromatography.

Where highly homogeneous serum albumin is used in HPLC, it is particularly preferred if the highly homogeneous serum albumin is immobilised on a silica matrix, typically an epoxy silica matrix, such as Kromasil 200 Å (5 μm) or Polygosil 300 Å (7 μm).

Enantioselective chromatography columns of the invention do not suffer the problems faced by prior art columns of significant variation of chromatographic performance, including variation in the retention of enantiomers and in enantioselectivity.

Variability of a column type can be determined by taking 10 columns of that type. To each column is added a racemic mixture of warfarin using 1-propanol/phosphate buffer (pH 7.5) 15/85 (v/v), 0.6 ml/min flow rate. The relatively unbound fraction (i.e. the R-enantiomer) is collected from the column and the recovery of the relatively bound (S—) enantiomer of warfarin is determined by UV absorption at 300 nm or 240 nm thereby to determine the capacity factor of the column and the enantioselectivity factor. Capacity factor (k′) is a measure of the time the solute spends in the stationary phase relative to the time it spends in the mobile phase and is calculated as k′=t_{R}−t_{M}/t_{M}, where t_{R} is the retention time of an injected analyte (e.g. S— or R-enantiomer of warfarin) and t_{M} is the time taken for the mobile phase to pass through the column. Enantioselectivity factor (α) describes the separation of two species (e.g. S— and R-enantiomers of warfarin) and is calculated as α=k′_{S}/k′_{R} where k′_{S} is the capacity factor of one species (e.g. the S-enantiomer of warfarin) and k′_{R} is the capacity factor of the other species (e.g. the R-enantiomer of warfarin).

Using the above method, enantioselection using prior art albumin columns is expected to produce a capacity factor varying by ±40-50%. By contrast, the use of highly homogeneous serum albumin according to the present invention can provide a lower level of variability, such as ±30%, ±20%, ±10% or less.

The standard deviation (σ) of a column type in this test can be calculated as follows

$$σ = \sqrt{\frac{{\sum }_{i = 1}^{10} {( {X}_{i} - \overline{X} )}^{2}}{10}}$$

wherein “X” is the capacity factor or the enantioselectivity factor of each column for the relatively bound enantiomer of warfarin and “i” is the sample number between 1 and 10.

Variability is expressed as relative standard deviation (RSD%) wherein
RSD%=100σ/\overline{x}
and can be applied to variability of capacity factor and/or enantioselectivity factor.

A column of the present invention will have a capacity factor and/or enantioselectivity factor RSD% less than that observed for an albumin column of the prior art when tested by a method as defined above, wherein each column type contains the same mass of immobilised albumin. Ideally, differences between highly homogeneous serum albumin and serum albumin of the prior art is determined using columns that have been prepared by the same technique, such as using the immobilisation technique exemplified below for Kromasil 200 Å, 5 μm.

A second aspect of the present invention provides an enantioselective chromatography column comprising, as the immobilised phase, highly homogeneous serum albumin. The column is preferably suitable for use in HPLC. Although any matrix to which albumin can be immobilised may be used, typically the column comprises an agarose matrix, a polymer (such as PSDB) matrix or, preferably, a silica matrix (typically an epoxy silica matrix, such as Kromasil 200 Å (5 μm) or Polygosil 300 Å (7 μm)), as an immobile phase to which the highly homogeneous serum albumin is affixed.

In a third aspect of the present invention, there is provided a process for selecting an enantiomer of a chiral compound, comprising the steps of
(i) exposing a mixture of enantiomers of the compound to highly homogenous serum albumin for a period to allow selective binding of one enantiomer to the albumin; and
(ii) separating the relatively unbound enantiomer from the albumin.

Preferably the highly homogeneous serum albumin used in a process according to the third aspect of the invention is immobilised. Even more preferably the highly homogeneous serum albumin used in a process according to the third aspect of the invention is immobilised in a column according to the second aspect of the invention. Yet more preferably the highly homogeneous serum albumin used in a process according to the third aspect of the invention is immobilised to a column suitable for use in HPLC.

Accordingly, using a process according to the third aspect of the invention, the skilled person can separate enantiomers of a chiral compound wherein those enantiomers interact differentially with albumin, such as those described above.

Thus in one embodiment the thus separated (relatively unbound) enantiomer is the enantiomer of interest.

In another embodiment, the enantiomer relatively bound by the albumin is the enantiomer of interest. In that case, a process of the third aspect of the invention preferably further comprises the step of separating the relatively bound enantiomer from the albumin.

Typically this further step is achieved by eluting the relatively bound enantiomer from the albumin using techniques well known in the art. For example, elution of the relatively bound enantiomer can be achieved by varying the environment of the highly homogeneous albumin by one or more parameter selected from ionic strength, pH, temperature, or the concentration of a displacing ligand (a ligand, different to the two enantiomers being separated, which competes for binding to the albumin). Elution of the relatively bound enantiomer may also be achieved using organic solvent modification of the albumin.

In either case, the enantiomer of interest can be obtained in a form that is enriched in one enantiomer, relative to the abundance of that enantiomer in the starting material.

Usually, where a substantially racemic mixture is separated, one enantiomer will represent at least 60% of the separated chiral compound obtained by the method of the third aspect of the invention. Typically, one enantiomer will represent at least 70%, more typically at least 80%, even more typically at least 90% of the separated chiral compound obtained by the method of the third aspect of the invention. In a preferred embodiment, one enantiomer will represent at least 95%, more preferably at least 98%, even more preferably at least 99%, yet more preferably at least 99.9%, most preferably, substantially 100% of the separated chiral compound obtained by the method of the third aspect of the invention.

If the mixture of enantiomers to be separated by the method of the third aspect of the invention is not substantially racemic, for example if the enantiomer of interest is represented at, say, less than 25%, 10%, 5%, 2% or 1% of the total chiral compound to be separated, then a process according to the third aspect of the invention may nevertheless be used to increase the relative abundance of the enantiomer of interest in the product of the process. Thus, where the mixture of enantiomers to be separated by the method of the third aspect of the invention is not substantially racemic, the enantiomer of interest may represent at least 1%, at least 5%, at least 10%, at least 20%, at least 30%, at least 40%, at least 50%, at least 60%, at least 70%, at least 80%, at least 90%, at least 95%, at least 98%, at least 99%, at least 99.5%, at least 99.9% or substantially 100% of the separated chiral compound obtained by the method of the third aspect of the invention.

In another aspect of the present invention, highly homogeneous serum albumin as defined above can be used in affinity chromatography. Albumins have previously been used in affinity chromatography, typically immobilised on a solid phase such as silica, sepharose or agarose (for example, see Simpson et al, 1980, J. Biol. Chem., 255(13), 6092-6097) Highly homogeneous serum albumin used in the context of affinity chromatography can reduce the variability of previous affinity chromatography techniques and hence provide a method that is better suited to regular and consistent use.

Accordingly, the present invention provides for the use of highly homogeneous serum albumin in affinity chromatography. The present invention also provides an affinity chromatography column comprising, as the immobilised phase, highly homogeneous serum albumin. An enantioselective chromatography column as defined above is also suitable for use as an affinity chromatography column. The present invention also provided a process for increasing the purity of an agent that binds to albumin, comprising the steps of (i) exposing a relatively impure preparation of the agent to an affinity chromatography column comprising, in the immobilised phase, highly homogeneous serum albumin, under conditions that favour the selective binding of the agent to the albumin; (ii) removing unbound components of the relatively impure preparation of the agent; and (iii) eluting the agent from the affinity chromatography column to obtain a preparation of the agent with increased purity. Thus an albumin-binding agent may be obtained in an enriched form.

The albumin-binding agent may be any agent that is specifically bound by albumin. Albumin can specifically bind numerous different agents. Typically, an albumin-binding agent is a hydrophobic anionic agent. For example, suitable agents are listed in Peters (1996, op. cit.: see particularly Chapter 3). Thus, the albumin-binding agent may be an amino acid or derivative thereof, an anti-albumin antibody, or a profen, like ibuprofen, fenoprofen, ketoprofen and the like, or any other suitable agent, such as an agent listed above as a chiral compound. Thus highly homogeneous serum albumin may be used not only as a chiral selector to separate one enantiomer of a chiral compound from another, but also in affinity chromatography to separate a chiral compound from other (i.e. chemically different) contaminating species.

Once an enantiomer or albumin-binding agent has been obtained in an enriched form using a method as defined above, that enantiomer or albumin-binding agent can be formulated with a pharmaceutically acceptable carrier or diluent.

A pharmaceutically acceptable carrier or diluent must be “acceptable” in the sense of being compatible with the enantiomer or albumin-binding agent and not deleterious to the recipients thereof. Typically, the carrier or diluent will be water or saline which will be sterile and pyrogen free.

An enantiomer or albumin-binding agent formulated with pharmaceutically acceptable carrier or diluent can be presented in a unit dosage form. For example, the formulation may be presented in a unit dosage form suitable for administration by any conventional method including oral and parenteral (eg subcutaneous or intramuscular) injection.

Formulations may conveniently be presented in unit dosage form and may be prepared by any of the methods well known in the art of pharmacy. Such methods include the step of bringing into association the active ingredient (compound of the invention) with the carrier, which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention may be suitable for oral administration and may be presented as discrete units such as capsules, cachets or tablets, each containing a predetermined amount of the enantiomer or albumin-binding agent; as a powder or granules; as a solution or a suspension in an aqueous liquid or a non-aqueous liquid; or as an oil-in-water liquid emulsion or a water-in-oil liquid emulsion. The enantiomer or albumin-binding agent may also be presented as a bolus, electuary or paste.

A tablet may be made by compression or moulding, optionally with one or more accessory ingredients. Compressed tablets may be prepared by compressing in a suitable machine the enantiomer in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the enantiomer in a flavoured basis, usually sucrose and acacia or tragacanth; pastilles comprising the enantiomer or albumin-binding agent in an inert basis such as gelatin and glycerin, or sucrose and acacia; and mouth-washes comprising the enantiomer in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an enantiomer or albumin-binding agent.

It should be understood that in addition to the ingredients particularly mentioned above the formulations that may be produced using the methods according to the third aspect of the invention may include other agents conventional in the art having regard to the type of formulation in question, for example those suitable for oral administration may include flavouring agents.

In a fourth aspect of the present invention, there is provided an enantiomer of a chiral compound obtainable by a method according to the third aspect of the invention.

The invention will now be described in more detail by reference to the following Examples.

### EXAMPLES

Purification of Recombinant Human Serum Albumin (rHA)

The rHA sample, obtained by expression of HSA gene in the yeast Saccharomyces cerevisiae, was supplied by Delta Biotechnology Limited (Castle Court, Nottingham, UK) under the name “RECOMBUMIN 20” in aqueous solution 20% (w/v) (145 mmol/L sodium chloride, 0.16 mmol sodium octanoate/g albumin and 15 mg/L Tween 80). The protein was defatted before use by a slightly modified version of Chen's charcoal procedure (Chen RF, 1967, J. Biol. Chem., 242, 173-181). The sample of rHA was dialysed against a KH₂PO₄ 10 mM/1-propanol 80/20 (v/v) mixture, with 0.5 g charcoal/g rHA, for 1 hour. The pH of the suspension was adjusted at pH 3 with HCl 0.1 N. Eight subsequent washings were performed decreasing progressively the propanol concentration from 20% to 1%. The last four dialysis processes were performed against phosphate buffer 10 mM at pH 3, 4.6, 7.4 and 7.4, respectively.

The success of the purification was checked by determining the binding properties of the protein for ligands that selectively bind at specific binding sites. In particular phenylbutazone, diazepam and bilirubin were used as markers. These compounds are known to bind selectively at site I, site II and site III on human serum albumin (Peters, 1996, “All About Albumin: biochemistry, genetics, and medical applications”, Academic Press, New York; Kragh-Hansen, 1990, Dan. Med. Bull., 37, 57-84; Sudlow et al, 1975, Mol. Pharmacol., 11, 824-832; Bertucci & Domenici, 2002, Current Medicinal Chemistry, 9, 1463-1481). The induced CD signal observed in the ligand binding to albumin was measured as difference between the spectrum of the drug-protein complex and the spectrum of the protein. The CD difference spectra were carried out in the region where the lowest energy electronic transition of the ligands occurs. This allowed analysis of the CD data at a wavelength where the albumin gives a negligible contribution, as in the case of phenylbutazone and diazepam, or it does not contribute at all, as in the case of bilirubin. The measurement of the CD difference spectra gives direct information on the enantioselectivity of the binding between the ligand and the protein, the observed CD signal being directly related to the amount of bound ligand (Ascoli et al, 1995, J. Pharm. Sci., 84, 737-741; Bertucci & Domenici, op.cit.).

Thus CD difference data were used to determine the binding parameters. In particular, the affinity constants of the drugs, at the high affinity binding sites, were determined by following the change of the intensity of the induced CD spectra upon the molar concentration of the 1/1 [drug/albumin] adduct. In this case, the CD technique is used to monitor stereoselective binding. In the case of the used markers the high affinity binding sites are stereoselective. We used progressive dilutions of the [rHA]/[drug] adduct in a 1/1 molar ratio. The intensity of the induced CD spectra decreases by decreasing the concentration of the complex, depending on the value of the dissociation constant. The K values obtained for the recombinant and the serum-derived human serum albumins are, for the high affinity binding sites, quite close for all the three markers.

Secondary Structure of rHA

The purified sample of rHA showed a secondary structure comparable with that of serum-derived human serum albumin, as evaluated by CD. Indeed the CD of the two proteins is almost superimposable in the high-energy portion of the spectrum (260-180 nm).

Immobilisation Of The Protein On The Silica Matrix

Silica gel was heated for 15 h at 180° C. and 1 mm Hg. 2 g of distilled (3-glycidoxypropyl)trimethoxysilane in 5 ml of dry xylene were added drop wise to 5.2 g of silica in 100 ml of dry xylene, and the mixture was refluxed for 36 h. After filtration on a fritted disk of porosity 4, the product was washed twice with dry xylene and dry acetone. The bonded silica was dried for 18 h at 40° C. under vacuum. The activated silica was then packed into 150×4.6 mm I.D. HPLC columns. The column was washed with 100 ml of acetone and dried at 70° C. under helium. A solution of the protein (10 mg/ml) in potassium phosphate buffer (0.05 M, pH 7.5) containing 2 M ammonium sulphate was circulated through the column, in closed circuit for 24 h. The column was then rinsed with 100 ml of 0.05 M KH₂PO₄ (pH 6.0) solution. After that, the column was flushed with 50 ml of 1 M glycine in 0.05 M KH₂PO₄ (pH 7.0) solution and then rinsed with 20 ml of 0.05 M KH₂PO₄ (pH 7.0) solution.

Before storage, 100 ml of potassium phosphate buffer (0.05 M, pH 7.5) solution containing 0.01% sodium azide was passed through the column.

The amount of the immobilised protein was determined by UV absorbance at 280 nm of the protein solution before and after the immobilisation procedure.

Immobilisation involves a reaction between an amino group of a lysine residue of the protein and the epoxide groups of the derivatised silica.

The effect of the identity of the silica gel immobile phase on method efficacy was investigated. Silica gel was heated for 15 h at 180° C. under 1 Pa. under argon, 15 g of silica gel were added to 120 ml of dry toluene and refluxed, then 2.8 g of distilled (3-glycidoxypropyl)trimethoxysilane in 10 ml of dry toluene were added drop wise and the mixture was refluxed for 12 h. After cooling and filtration on a fritted disk, the product was washed twice with dry toluene and dry acetone. The bonded silica was dried for 15 h at 80° C. under vacuum (1 Pa).

2.1 g of activated silica were suspended in 40 ml of distilled and dry acetonylacetone mixed for 2 min and sonicated for a further two minutes. The mixture was put in the packing tank and pumped into a 150×4.6 mm I.D. HPLC column with 150 ml of dry acetone under a pressure of 40,000 kPa (400 bar). The column was washed with 100 ml of dry acetone and dried at 80° C. under helium for 16 hours. A solution of the protein (10 mg/ml) in potassium phosphate buffer (0.05 M, pH 7.5) containing 2 M ammonium sulphate was circulated through the column, in closed circuit for 24 h.

The column was then rinsed with 100 ml of solution A (KH₂PO₄+K₂HPO₄, 0.05 M pH=6.7). Then 4.5 ml of HSA was added to a solution containing KH₂PO₄+K₂HPO₄, 0.05 M and (NH₄)₂SO₄1.5 M (pH=6.0). The mixture was flushed into the column in closed circuit. The column was backflushed every 15 min for 1 hour and every 30 min for the next 3 hours. After that, the column was rinsed with 90 ml of solution A and was flushed with 30 ml of solution A containing 1 M of glycine. Then the column was rinsed with 30 ml of solution A and a 30 ml of solution A containing 0.01% in weight of NaN₃ was passed through the column. The column was then stored in a fridge.

The amount of the immobilised protein was determined by UV absorbance at 280 nm of the protein solution before and after the immobilisation procedure. The amount of immobilisation (mg) per gram of epoxy silica were determined to be as follows:
Kromasil 200 Å, 5 μm: 115 (±5).
Polygosil 300 Å, 7 μm: 31, 62, 59, 25, 43.

These results show that the immobilisation is better on Kromasil (specific surface>200 m² .g⁻¹) than on Polygosil (specific surface<100 m².g⁻¹). It also appears that Kromasil is more homogeneous than Polygosil. Our results for Kromasil have always been between 110 and 120, where as the is results for Polygosil in five separate columns are more variable. Therefore, the use of Kromasil 200 Å, 5 μm is preferred to Polygosil 300 Å, 7 μm.

Enantioselective HPLC Analysis by the rHA-Based Column

The rHA-based column resulted in the efficient resolution of a variety of chiral drugs and amino acid derivatives. As an example enantiomeric resolution was obtained for rac-warfarin (α=2.1), rac-lorazepam hemisuccinate (α=5.3), N-benzoyl-DL-leucine (α=2.8), using 1-propanol/phosphate buffer (pH 7.5) 15/85 (v/v), 0.6 ml/min flow rate. The obtained values of enantioselectivity are comparable or higher with respect to those obtained with the corresponding HSA-based columns, under the same experimental conditions, but the levels of variability between columns is lower than that observed in the prior art.

1-11. (canceled)

12. A method for selecting an enantiomer of a chiral compound, comprising the steps of
(i) exposing a mixture of enantiomers of the compound to highly homogenous serum albumin for a period to allow selective binding of one enantiomer to the albumin; and
(ii) separating the relatively unbound enantiomer from the albumin.

13. The method according to claim 12 wherein the highly homogeneous serum albumin is immobilised.

14. The method according to claim 13 wherein the immobilised highly homogeneous serum albumin is immobilised on an enantioselective chromatography column.

15. The method according to claim 12 further comprising the step of separating the relatively bound enantiomer from the albumin.

16. The method according to claim 12 wherein the chiral compound is selected from an amino acid, an amino acid derivative, a sulfoxide, a sulfoxamine derivative, a racemic amine, a non-steroidal anti-inflammatory drug, an aryl propionate anti-inflammatory drug, an N-methylated barbiturate a benzodiazepine, a profen and a coumarin.

17. The method according to claim 12 wherein the chiral compound is selected from warfarin, warfarin metabolites such as 6- and 7-hydroxywarfarin and warfarin alcohols, lorazepam (e.g. lorazepam hemisuccinate), oxazepam, temazepam, N-benzoyl-DL-leucine, tryptophan, benzoin, eprisone, chlorphemramine, kynurenine, prilocaine, promethazine, donepezil and its salts (e.g. donepezil hydrochloride, sold under the trade name Asicept), thiopenfal, ibuprofen, naproxen, ketoprofen, suprofen or fenoprofen and salts of these.

18. A method for increasing the purity of an agent that binds to albumin, comprising the steps of
(i) exposing a relatively impure preparation of the agent to an affinity chromatography column comprising, in the immobilised phase, highly homogeneous serum albumin, under conditions that favour the selective binding of the agent to the albumin;
(ii) removing unbound components of the relatively impure preparation of the agent; and
(iii) eluting the agent from the affinity chromatography column to obtain a preparation of the agent with increased purity.

19. The method according to claim 12 further comprising the step of formulating a separated enantiomer or agent with increased purity with a pharmaceutically acceptable carrier or diluent, thereby to produce a pharmaceutical preparation.

20. The method according to claim 17 further comprising the step of presenting the pharmaceutical preparation in a unit dosage form.

21. The method according to claim 12 wherein the highly homogeneous serum albumin is highly homogeneous human serum albumin.

22. The method according to claim 12 wherein the highly homogeneous serum albumin is highly homogeneous recombinant serum albumin.

23. An enantiomer of a chiral compound obtainable by a method according to claim 12.

24. A method substantially as described herein with reference to the examples.

25. The method of claim 14 wherein the enantioselective chromatography column comprises an immobilised phase comprising highly homogeneous serum albumin.

26. The method of claim 14 wherein the enantioselective chromatography column is used for HPLC.

27. The method of claim 14 wherein the enantioselective chromatography column comprises highly homogeneous serum albumin immobilised on a silica matrix.
